(19) United States
(12) Plant Patent Application Publication
(10) Pub. No.: US 2026/0240061 P1
(43) Pub. Date: Aug. 13, 2026

(54) CANNABIS PLANT NAMED '4D2'

(50) ;

(71) Applicant: ESB LLC, Willits, CA (US)

(72) Inventor: KATIE JEANE, WILLITS, CA (US)

(21) Appl. No.: 19/431,253

(22) Filed: Dec. 23, 2025

Related U.S. Application Data
(60) Provisional application No. 63/738,293, filed on Dec. 23, 2024.

(51) Int. Cl. A01H 6/28; A01H 5/00

(52) U.S. Cl. CPC A01H 6/28; A01H 5/00
USPC PLT/263.1

(57) ABSTRACT

The unique annual herbaceous Cannabis plant variety ‘4D2’ is provided. The variety is a selection resulting from a sexual cross. The variety can be distinguished by its outstanding features of unique cannabinoid profile containing THC, CBD, THCV and CBDV.

### CROSS REFERENCE TO RELATED APPLICATIONS

The present Application for Patent claims benefit of U.S. Provisional Application No. 63/738,293 entitled “CANNABIS PLANTS NAMED ‘HC-13’, ‘HR-16’ ‘RQ5’, ‘4D2’, AND ‘ESBCBG-1’” filed Dec. 23, 2025, which is hereby expressly incorporated by reference herein.

Latin name of the genus and species: Genus—Cannabis. Species—sativa.

Variety denomination: The new Cannabis plant claimed is of the variety denominated ‘4D2’.

### BACKGROUND OF THE INVENTION

### Field of the Invention

The present invention relates to a new and distinct annual variety of C. sativa, which has been given the variety denomination of ‘4D2’. ‘4D2’ is intended for use as medicinal herb for sale in Cannabis dispensaries, home garden plant, hemp farming, high value cannabinoid extraction and to make products containing THC, CBD, THCV and CBDV. This variety also has value as part of a breeding program to develop improved Cannabis varieties.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments and is also the active ingredient in the FDA-approved drug Epidiolex®. CBD is just one of at least dozens—perhaps hundred—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1;20(11):2083-2085; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Non-hemp forms of Cannabis, frequently referred to as marijuana, have been legalized for medicinal use in many states and for recreational use (sometimes called “adult use”) in a growing number of states. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘4D2’. ‘4D2’ is intended for use as medicinal herb for sale in Cannabis dispensaries, home garden plant, hemp farming, high value cannabinoid extraction, and to make products containing THC, CBD, THCV and CBDV. This variety also has value as part of a breeding program to develop improved Cannabis varieties, etc.

The new C. sativa variety ‘4D2’ is a selection resulting from a sexual cross of C. sativa plants at Willits, CA, involving a seed parent known as ‘HR-16’ and a pollen parent known as ‘Pink Boost Goddess’.

‘4D2’ offers a unique blend of THC, CBD, THCV and CBDV compared to its parents and closely related cultivars.

The selection was subsequently evaluated for 4 years at Willits, CA.

The new variety reproduces true to type with all of the characteristics, as herein described, firmly fixed and retained through successive generations of asexual propagation.

Asexual reproduction of the new variety by cutting propagation since 2020 at Willits, CA has demonstrated that the new variety reproduces true to type with all of the morphological characteristics, as herein described, firmly fixed and retained through successive generations of such asexual propagation. Chemotypic characteristics of each new variety are variable based upon cultivation conditions, as is typical of Cannabis plants. Accordingly, while chemotypic information provided herein is representative of performance of the new varieties under a particular set of cultivation conditions, it is not limiting on other chemotypic profiles obtainable under a different set of cultivation conditions. A stable seed line of each of the new varieties has also been developed by successive generations of inbreeding and selection to correspond to the phenotypic characteristics described herein. A stable seed line of the new variety has also been developed by successive generations of inbreeding and selection to correspond to the phenotypic characteristics described herein.

The following characteristics of the new variety have been repeatedly observed and can be used to distinguish ‘4D2’ as a new and distinct variety of C. sativa plant: unique cannabinoid profile containing THC, CBD, THCV and CBDV.

### BRIEF DESCRIPTION OF THE DRAWINGS

The accompanying photographic illustrations show the typical appearance of the new variety ‘4D2’. The colors are as nearly true as is reasonably possible in a color representation of this type. Colors in the photographs may differ slightly from the color values cited in the detailed botanical description which accurately describes the colors of the new plant.

FIG. 1 is a photograph of the new variety ‘4D2’, demonstrating a closeup of the inflorescence.

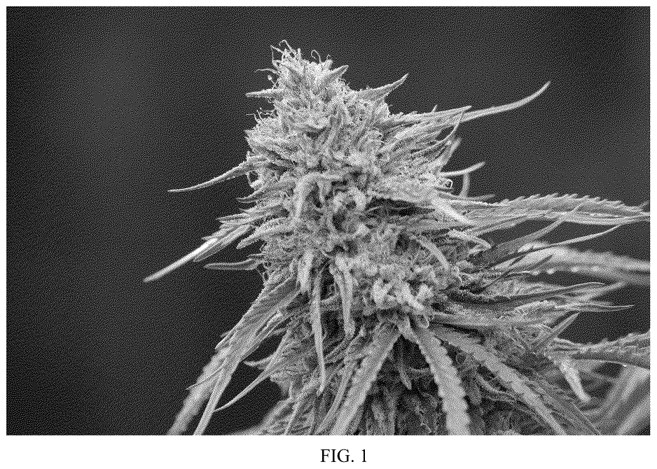

FIG. 2 is a photograph of the new variety ‘4D2’, demonstrating the growth habit.

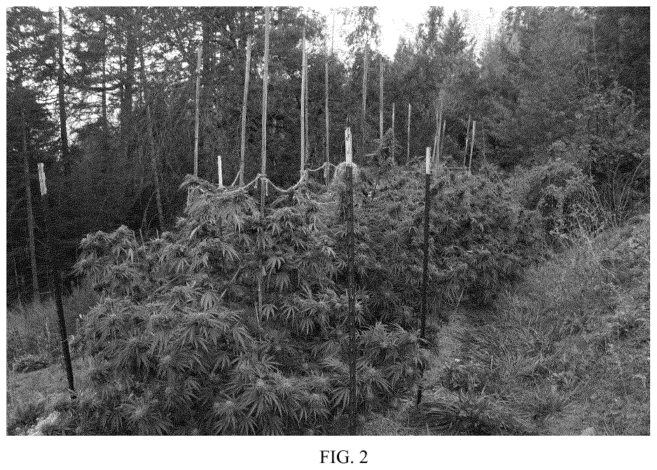

### DETAILED BOTANICAL DESCRIPTION

The following detailed description sets forth the distinctive characteristics of ‘4D2’. The datum which defines these characteristics was collected from asexual reproductions of the original selection. Dimensions, sizes, colors, and other characteristics are approximations and averages set forth as accurately as possible.
Type: Herbaceous annual.
Classification:
   a. Family.—Cannabaceae.
   b. Genus.—Cannabis.
   c. Species.—sativa.
   d. Common name.—Marijuana.
Parentage: ‘HR-16’ x ‘Pink Boost Goddess’.

1. A new and distinct variety of Cannabis plant named ‘4D2’, as illustrated and described herein.
